(19) United States
(12) Patent Application Publication
(10) Pub. No.: US 2025/0099315 A1
(43) Pub. Date: Mar. 27, 2025

(54) EXTREMITY DRAINAGE DEVICES, SYSTEMS, AND METHODS OF USE THEREOF

(71) Applicant: Loma Linda University, Loma Linda, CA (US)

(72) Inventor: Rebecca RAJFER, Loma Linda, CA (US)

(73) Assignee: Loma Linda University, Loma Linda, CA (US)

(21) Appl. No.: 18/728,679

(22) PCT Filed: Jan. 12, 2023
(86) PCT No.: PCT/US2023/010655

§ 371 Date: Jul. 12, 2024

Related U.S. Application Data
(60) Provisional application No. 63/298,694, filed on Jan. 12, 2022.

(51) Int. Cl. A61G 13/10; A61M 1/00

(52) U.S. Cl. CPC A61G 13/102; A61M 1/84

(57) ABSTRACT

An extremity drainage device, system, and method of use thereof are disclosed. The drainage device may be configured to collect a bodily fluid of a patient and/or a related irrigation fluid during a medical procedure. The drainage device may include a receptacle having a bottom surface and an outer wall extending upward from a perimeter of the bottom surface to an edge, the bottom surface and the outer wall defining a cavity. The edge may include a plurality of upper edge portions that substantially lie in a plane transverse to the outer wall and a plurality of lower edge portions, each lower edge portion connected to two upper edge portions. In addition, the lower edge portions may define in the outer wall at least two diametrically opposed side openings, each of the side openings having at least a partially curved profile at a bottom-most portion of the lower edge portion.

### CROSS REFERENCE TO RELATED APPLICATION

This application claims the benefit of U.S. Provisional Application No. 63/298,694, filed on Jan. 12, 2022, which is incorporated herein by reference.

### FIELD OF THE INVENTION

This application relates to extremity drainage devices, systems, and methods of use thereof. In particular, this application relates to extremity drainage pans configured to collect bodily fluids and systems and methods of use thereof.

### BACKGROUND OF THE INVENTION

Emesis basins, also known as kidney dishes, and rectangular pans, also known as lasagna pans, have been used in clinical settings to collect bodily fluids. During procedures involving the extremities, such as a surgery of a leg or an arm, or a pre-or post-operative procedure, such as cleaning the wound and/or changing the dressings, a kidney dish or lasagna pan may be positioned against or under the extremity to collect bodily fluids that drain out of the wound.

Gross spillage of irrigation fluids occurs at least due to a mismatch of the extremity shape (i.e., round or conical arm or leg) and the shape of the drainage pan or dish used to collect the fluids as they flow out of the wound. Overflow of the irrigation fluid containing bodily fluids, including blood and pus, may collect in the surgical drapes creating a potentially contaminated pool in the sterile field, on the floor creating a hazardous work environment, and onto the surgeon's clothing creating an infection control situation. Pooling of the bodily fluid in the drapes may require additional vital anesthetic time or costly operating room time while the surgeons attempt to collect the fluid by soaking it with towels or sponges or vacuuming it with suction tubing.

In view of the above-discussed issues, a need exists for a drainage pan device that reduces spillage of bodily fluids.

### SUMMARY OF THE INVENTION

In a first exemplary embodiment of the present invention, a drainage device is disclosed. The drainage device may be configured to collect a bodily fluid of a patient and/or a related irrigation fluid during a medical procedure. The drainage device may include a receptacle having a bottom surface and an outer wall extending upward from a perimeter of the bottom surface to an edge, the bottom surface and the outer wall defining a cavity. The edge may include a plurality of upper edge portions that substantially lie in a plane transverse to the outer wall and a plurality of lower edge portions, each lower edge portion connected to two upper edge portions. In addition, the lower edge portions may define in the outer wall at least two diametrically opposed side openings, each of the side openings having at least a partially curved profile at a bottom-most portion of the lower edge portion.

In some versions of the first embodiment, the drainage device may include a drain opening configured to be connected to tubing for draining the collected fluid out of the receptacle. The lower edge portions may define in the outer wall at least two pairs of diametrically opposed side openings, each of the side openings having at least a partially curved profile at a bottom-most portion of the lower edge portion. Also, the outer wall may include at least two planar sides and the lower edge portions define in the planar sides the at least two diametrically opposed side openings. The outer wall may include at least two planar sides and the lower edge portions define in the planar sides one pair of the diametrically opposed side openings. Additionally, a side opening in one of the at least two pairs of side openings may have a differently shaped or sized opening than a side opening in a different one of the at least two pairs of side openings. Both side openings in each of the at least two pairs of side openings may have substantially the same shaped and sized opening.

In a second exemplary embodiment of the present invention, a drainage assembly is disclosed. The drainage assembly may be configured to collect a bodily fluid of a patient and/or a related irrigation fluid during a medical procedure. The drainage assembly may include a receptacle having a bottom surface and an outer wall extending upward from a perimeter of the bottom surface to an edge, the bottom surface and the outer wall defining a cavity. The edge may include a plurality of upper edge portions that substantially lie in a plane transverse to the outer wall and a plurality of lower edge portions, each lower edge portion connected to two upper edge portions. The drainage assembly may include a drain opening positioned on or near the bottom surface and a suction pump operatively connected to the drain opening and configured to help drain the collected fluid out of the receptacle. In addition, the lower edge portions of the edge may define in the outer wall at least two diametrically opposed side openings, each of the side openings having at least a partially curved profile at a bottom-most portion of the lower edge portion.

In some versions of the second exemplary embodiment, the lower edge portions may define in the outer wall at least two pairs of diametrically opposed side openings, each of the side openings having at least a partially curved profile at a bottom-most portion of the lower edge portion. Also, the outer wall may include at least two planar sides and the lower edge portions define in the planar sides the at least two diametrically opposed side openings. The outer wall may include at least two planar sides and the lower edge portions define in the planar sides one pair of the diametrically opposed side openings. Additionally, a side opening in one of the at least two pairs of side openings may have a differently shaped or sized opening than a side opening in a different one of the at least two pairs of side openings. Both side openings in each of the at least two pairs of side openings may have substantially the same shaped and sized opening.

In a third exemplary embodiment of the present invention, a method for collecting a bodily fluid of a patient and/or a related irrigation fluid with a drainage device is disclosed. The method may include providing the drainage device. The drainage device may include a receptacle having a bottom surface and an outer wall extending upward from a perimeter of the bottom surface to an edge, the bottom surface and the outer wall defining a cavity. The edge may include a plurality of upper edge portions that substantially lie in a plane transverse to the outer wall and a plurality of lower edge portions, each lower edge portion connected to two upper edge portions. In addition, the lower edge portions may define in the outer wall at least two diametrically opposed side openings, each of the side openings having at least a partially curved profile at a bottom-most portion of the lower edge portion. The method may also include positioning a portion of the patient within at least one of the side openings of the drainage device and collecting, with the drainage device, the bodily fluid and/or the related irrigation fluid.

In some versions of the third exemplary embodiment, the method may include sterilizing the drainage device. In addition, the lower edge portions may define in the outer wall at least two pairs of diametrically opposed side openings, each of the side openings having at least a partially curved profile at a bottom-most portion of the lower edge portion. Also, the outer wall may include at least two planar sides and the lower edge portions define in the planar sides the at least two diametrically opposed side openings. The outer wall may include at least two planar sides and the lower edge portions define in the planar sides one pair of the diametrically opposed side openings. Additionally, a side opening in one of the at least two pairs of side openings may have a differently shaped or sized opening than a side opening in a different one of the at least two pairs of side openings. Both side openings in each of the at least two pairs of side openings may have substantially the same shaped and sized opening.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a partial perspective and partial side diagram view of a drainage system according to an exemplary embodiment of the present disclosure.

FIG. 2 is a perspective view of a drainage device according to an exemplary embodiment of the present disclosure.

### DETAILED DESCRIPTION

Reference will now be made in detail to the exemplary embodiments, which are illustrated in the accompanying drawings. Wherever possible, the same reference numbers will be used throughout the drawings to refer to the same or like parts.

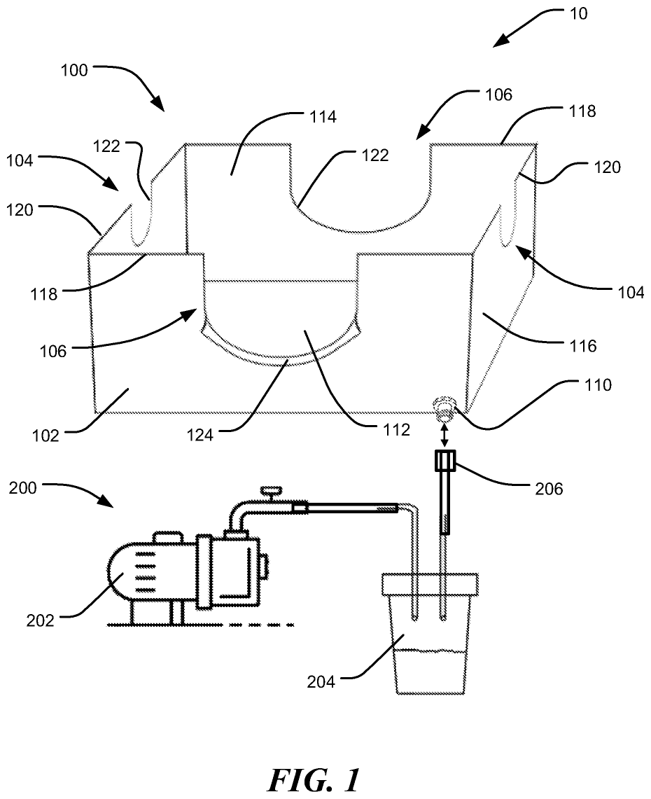

Described herein are extremity drainage devices, systems, and methods of use thereof. FIG. 1 depicts an exemplary embodiment of an extremity drainage assembly 10. The extremity drainage assembly 10 generally includes a drainage device 100 for collecting bodily fluids of a patient and/or related irrigation fluids during a medical procedure and a suction pump assembly 200 removably connected to the drainage device 100 for assisting with draining the collected bodily fluids.

The bodily fluids may include, for example, amniotic fluid, aqueous humour, bile, blood plasma, breast milk, cerebrospinal fluid, cerumen, chyle, exudates, gastric juice, lymph, mucus, pericardial fluid, peritoneal fluid, pleural fluid, pus, saliva, sebum, serous fluid, semen, sputum, synovial fluid, sweat, tears, urine, and vomit. Irrigation fluids may include, for example, water, saline solutions, antibiotics, surfactants, and antiseptics, among other medicaments.

As shown in FIG. 1, the drainage device 100 includes a receptacle 102 having a bottom surface 112 and an outer wall 114 extending upward from a perimeter of the bottom surface 112 to an edge 118. The bottom surface 112 and the outer wall 114 collectively define a cavity therebetween. The bottom surface 112 may be substantially flat and have a polygonal perimeter. As shown in FIG. 1, the bottom surface 112 has a rectangular perimeter and, accordingly, the outer wall 114 has four planar sides 116. In other embodiments not shown, the bottom surface 112 may have a curvilinear perimeter, such as an ellipse, oval, circle, or kidney-shaped, resulting in the outer wall 114 having a single side wall. One having ordinary skill in the art would appreciate that in other embodiments the bottom surface 112 may have perimeter with 3 sides or more than 4 sides. The planar sides 116 may be generally rectangular, and as shown in FIG. 1, the planar sides 116 may have a slight draft angle to them such that they are slightly trapezoidal. Having a slight draft angle may permit two or more drainage devices 100 to stack on top of each other for reducing bulk storage volume.

The outer wall 114 (and planar sides 116) may extend to an upward facing edge 118. The edge 118 may provide a continuous pathway circumscribing and defining a generally downward opening into the receptacle 102. The edge 118 may comprise a plurality of upper edge portions 120 that may lie substantially within a plane and/or each may have substantially linear profiles. Each of the upper edge portions 120 may connect to another upper edge portion 120 in a corner where two planar sides 116 meet. Each of the upper edge portions 120 may also connect to a lower edge portion 122 at an opposite end from where it connects to an upper edge portion 120. The lower edge portion 122 may connect to upper edge portions 120 at both of its ends and may extend beneath the plane common to the upper edge portions 120. By extending below the upper edge portions 120, the lower edge portions 122 may define side openings 104, 106. The lower edge portions 122 may have linear and/or curvilinear sections. For example, as shown in FIG. 1, the lower edge portions 122 may have linear sections along the sides of the side openings 104, 106 and a curvilinear section or a curved profile along the bottom-most portion thereof, forming U-shaped side openings 104, 106. In other embodiments, the side openings 104, 106 may be circular, elliptical, oval, or any other curved profile. The size and shape of the side openings 104, 106 may be configured to comfortably fit an extremity of a patient in the prone, supine, or lateral recumbent positions. For example, the opening may be sized and shaped to fit the front, back, or side of a lower leg, upper leg, lower arm, or upper arm. In other embodiments, the opening may be sized and shaped to fit other parts of the body including shoulders, chest, etc.

The side openings 104, 106 may include two first side openings 104 diametrically opposed to one another on oppositely facing planar sides 116 and two second side openings 106 diametrically opposed to one another on different oppositely facing planar sides 116. The first side openings 104 and the second side openings 106 may have differently sized and/or shaped profiles. The spacing between the first side openings 104 may be the same as the spacing between the second side openings 106 or the spacings may be different. For example, the first side openings 104 may be sized, shaped, and spaced apart to fit a patient's lower leg, lower arm, and upper arm, whereas the second side opening 106 may be sized, shaped, and spaced apart to fit a patient's upper leg. As another example, the first side openings 104 may be sized, shaped, and spaced apart to fit a smaller patient's (such as a child or smaller adult) upper leg, whereas the second side opening 106 may be sized, shaped, and spaced apart to fit a larger patient's (such as a large adult) upper leg. One having ordinary skill in the art would appreciate the various different combination of sizes, shapes, and spacings for which the side openings 104, 106 may be configured. As shown in FIG. 1, the first side openings 104 have the same general shape as the second side openings 106 but are smaller and spaced further apart than the second side openings 106.

FIG. 1 shows both of the first side openings 104 as having the same size and shape as each other and both of the second side openings 106 as having the same size and shape as each other, in other embodiments, the shapes and sizes of the openings within a pair may be different from each other. For example the one of the first side openings 104 (or second side opening 106) may be larger than the other first side opening 104 (or second side opening 106) for accommodating a proximal portion compared to a distal portion of the patient's extremity.

The side openings 104, 106 may include rounded surfaces to increase the contact surface for helping to distribute the weight of the extremity over a larger area and to ensure a better seal between the extremity and the drainage device 100. The rounded surface may entail the planar sides 116 having a radiused edge 118 or it may entail the side openings 104, 106 having a shelf or flange surface 124 along the lower edge portions 122. The shelf 124 may be flat or curved and may be present at the bottom portion of the lower edge portions 122 or along the entire lower edge portions 122. As shown in FIG. 1, the second side openings 106 include a shelf 124 that may be formed by rolling a portion of the planar sides 116 adjacent the lower edge portions 122 outward. In other embodiments not shown, both the first and the second side openings 104, 106 may include shelfs 124. The shelfs may alternatively be rolled inward or attached by gluing, welding, screwing, taping, etc. to the planar sides 116. Also, in other embodiments, the side openings 104, 106 may include a resilient gasket insert positioned on the lower edge portions 122.

The receptacle 102 may be comprised of a metallic material, such as stainless steel, or may be comprised of polymers or composites. Accordingly, such materials may lend the drainage device to be sterilized for reuse or disposed for single use.

The drainage device 100 may include a drain opening 110 configured to be connected to tubing for draining collected fluid out of the receptacle 102. The drain opening 110 may be positioned on the bottom surface 112 or on the outer wall 114 near the bottom surface 112. The drain opening 110 may be hole in the outer wall 114 or may include a nozzle protruding outward from the outer wall 114. The nozzle may be have smooth or threaded internal and/or external surfaces. For example, as shown in FIG. 1, the drain opening 110 includes an male Luer fitting having external threads and configured for receiving a female Luer fitting 206. In other embodiments, the drain opening may include a barbed nozzle or other tubing and hose connectors. The drain opening 110 may include a plug (not shown) for closing the drain opening 110 when it is not in use or for when it is in use but temporarily not configured to be drained.

As mentioned above, the drain opening 110 is configured to be connected to a Luer fitting 206, but in other embodiments, the drain opening 110 may be configured to be connected to other connectors or directly to tubing. In either configuration, the tubing may be routed to a fluid collection device 204 for removing and safely storing fluid drained from the drainage device 100. The fluid collection device 204 may be in fluid communication with a pump 202 for applying a vacuum and assisting with the draining of the fluid from the drainage device 100. The fluid collection device 204 may be positioned lower than the drainage device 100 such that gravity may assist the draining of the drainage device 100. The fluid collection device 204 may also be positioned lower than the pump 202 so as to prevent fluid from flowing to the pump 202.

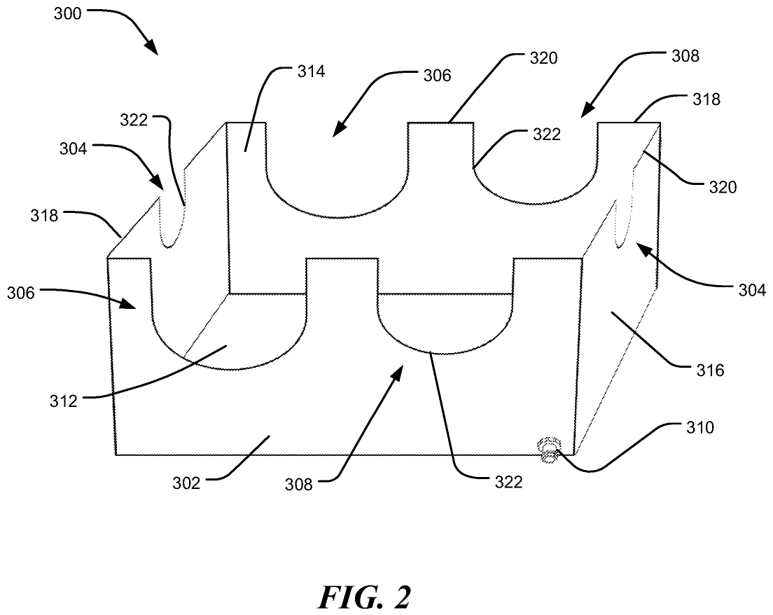

FIG. 2 shows another exemplary embodiment of a drainage device 300. The drainage device 300 in substantially the same as the drainage device 100 described above and shown in FIG. 1 with reference designators incremented by 200 for similar elements. This includes but is not limited to a receptacle 302, a drain opening 310, a bottom surface 312, an outer wall 314, one or more edges 318. Further, The edge 318 may comprise a plurality of upper edge portions 320 that may lie substantially within a plane and/or each may have substantially linear profiles. Each of the upper edge portions 320 may connect to another upper edge portion 320 in a corner where two planar 316 sides meet. Each of the upper edge portions 320 may also connect to a lower edge portion 322 at an opposite end from where it connects to an upper edge portion 320. The drainage device 300 may have some differences from the drainage device 100 such as including planar walls 316 having first side openings 304 and planar walls 316 having both second side openings 306 and third side openings 308. The first, second, and third side openings 304, 306, 308 may be differently sized and shaped in a similar manner as the first and second side openings 104, 106 described above. Moreover, the drainage device 300 may have more than three pairs of side openings, such as four pair of side openings (not shown), with each planar side wall 316 including two pairs of side openings. In other embodiments not shown, the drainage device 300 may have a hexagonal bottom surface with six planar sides extending upward from a perimeter thereof. In such embodiment, the drainage device 300 may have three pairs of diametrically opposed openings, with each opening positioned on its own planar side.

In an exemplary method of use, a practitioner may provide the drainage device 100 to a patient before a procedure is performed on the patient. The practitioner may connect the drainage device 100 to a suction pump assembly 200. The practitioner may position a portion of the patient within at least one of the side openings 104, 106 of the drainage device 100. The practitioner may position a portion of the patient within another one of the side openings 104, 106 of the drainage device 100. The practitioner may then perform a procedure on the patient leading to the need to collect bodily fluid and/or related irrigation fluid. The drainage device may then collect the bodily fluid and/or the related irrigation fluid. The suction pump assembly 200 may then drain the collected bodily fluid and/or the related irrigation fluid from the drainage device 100. Next, practitioner may clean and sterilize the drainage device 100 and then stack the drainage device 100 within the cavity of another drainage device 100.

It should be understood from the foregoing that, while particular aspects have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A drainage device configured to collect a bodily fluid of a patient and/or a related irrigation fluid during a medical procedure, the drainage device comprising:
a receptacle having a bottom surface and an outer wall extending upward from a perimeter of the bottom surface to an edge, the bottom surface and the outer wall defining a cavity; and
the edge including a plurality of upper edge portions that substantially lie in a plane transverse to the outer wall and a plurality of lower edge portions, each lower edge portion connected to two upper edge portions;
wherein the lower edge portions define in the outer wall at least two diametrically opposed side openings, each of the side openings having at least a partially curved profile at a bottom-most portion of the lower edge portion.

2. The drainage device of claim 1, further including a drain opening configured to be connected to tubing for draining the collected fluid out of the receptacle.

3. The drainage device of claim 1, wherein the lower edge portions define in the outer wall at least two pairs of diametrically opposed side openings, each of the side openings having at least a partially curved profile at a bottom-most portion of the lower edge portion.

4. The drainage device of claim 1, wherein the outer wall includes at least two planar sides and the lower edge portions define in the planar sides the at least two diametrically opposed side openings.

5. The drainage device of claim 3, wherein the outer wall includes at least two planar sides and the lower edge portions define in the planar sides at least one of the pairs of the diametrically opposed side openings.

6. The drainage device of claim 3, wherein a side opening in one of the at least two pairs of side openings has a differently shaped or sized opening than a side opening in a different one of the at least two pairs of side openings.

7. The drainage device of claim 6, wherein both side openings in each of the at least two pairs of side openings have substantially the same shaped and sized opening.

8. A drainage assembly configured to collect a bodily fluid of a patient and/or a related irrigation fluid during a medical procedure, the drainage assembly comprising:
a receptacle having a bottom surface and an outer wall extending upward from a perimeter of the bottom surface to an edge, the bottom surface and the outer wall defining a cavity;
the edge including a plurality of upper edge portions that substantially lie in a plane transverse to the outer wall and a plurality of lower edge portions, each lower edge portion connected to two upper edge portions;
a drain opening positioned on or near the bottom surface; and
a suction pump operatively connected to the drain opening and configured to help drain the collected fluid out of the receptacle;
wherein the lower edge portions define in the outer wall at least two diametrically opposed side openings, each of the side openings having at least a partially curved profile at a bottom-most portion of the lower edge portion.

9. The drainage assembly of claim 8, wherein the lower edge portions define in the outer wall at least two pairs of diametrically opposed side openings, each of the side openings having at least a partially curved profile at a bottom-most portion of the lower edge portion.

10. The drainage assembly of claim 8, wherein the outer wall includes at least two planar sides and the lower edge portions define in the planar sides the at least two diametrically opposed side openings.

11. The drainage assembly of claim 9, wherein the outer wall includes at least two planar sides and the lower edge portions define in the planar sides at least one pair of the diametrically opposed side openings.

12. The drainage assembly of claim 9, wherein a side opening in one of the at least two pairs of side openings has a differently shaped or sized opening than a side opening in a different one of the at least two pairs of side openings.

13. The drainage assembly of claim 12, wherein both side openings in each of the at least two pairs of side openings have substantially the same shaped and sized opening.

14. A method for collecting a bodily fluid of a patient and/or a related irrigation fluid with a drainage device, the method comprising:
providing the drainage device, the drainage device comprising:
a receptacle having a bottom surface and an outer wall extending upward from a perimeter of the bottom surface to an edge, the bottom surface and the outer wall defining a cavity; and
the edge including a plurality of upper edge portions that substantially lie in a plane transverse to the outer wall and a plurality of lower edge portions, each lower edge portion connected to two upper edge portions;
wherein the lower edge portions define in the outer wall at least two diametrically opposed side openings, each of the side openings having at least a partially curved profile at a bottom-most portion of the lower edge portion;
positioning a portion of the patient within at least one of the side openings of the drainage device; and
collecting, with the drainage device, the bodily fluid and/or the related irrigation fluid.

15. The method of claim 14, further comprising sterilizing the drainage device.

16. The method of claim 14, wherein the lower edge portions define in the outer wall at least two pairs of diametrically opposed side openings, each of the side openings having at least a partially curved profile at a bottom-most portion of the lower edge portion.

17. The method of claim 14, wherein the outer wall includes at least two planar sides and the lower edge portions define in the planar sides the at least two diametrically opposed side openings.

18. The method of claim 16, wherein the outer wall includes at least two planar sides and the lower edge portions define in the planar sides one pair of the diametrically opposed side openings.

19. The method of claim 16, wherein a side opening in one of the at least two pairs of side openings has a differently shaped or sized opening than a side opening in a different one of the at least two pairs of side openings.

20. The method of claim 19, wherein both side openings in each of the at least two pairs of side openings have substantially the same shaped and sized opening.
